SEQUENCE LISTING (9 sequences)

SEQ ID NO: 1 - protein, 76 aa, Artificial Sequence
  2D-HBN_Homo
  MISC_FEATURE (1)..(4): Optional residues
  MISC_FEATURE (76)..(76): Optional residue
GELTDIILKLIKSLQTQKLLAERLKTLLKVLEISQDSGADDKQVKKLLDEIRKLVEKIEKLARKQTKLVEKLLKKD

SEQ ID NO: 2 - protein, 74 aa, Artificial Sequence
  2D-HP_Homo
  MISC_FEATURE (1)..(3): Optional residues
SRTMYIRALEQSLREQEELAKRLKELLRELERLQREGSSDRDVKVLLWEIEALVEEIEKLARLQKELVEKLKRQ

SEQ ID NO: 3 - protein, 150 aa, Artificial Sequence
  2D-HP
SRTMYIRALEQSLREQEELAKRLKELLRELERLQREGSSDRDVKVLLWEIEALVEEIEKLARLQKELVEKLKRQGSGNMYIRALEQSLREQEELAKRLKELLRELERLQREGSSDRDVKVLLWEIEALVEEIEKLARLQKELVEKLKRQD

SEQ ID NO: 4 - protein, 151 aa, Artificial Sequence
  2D-HBN
GELTDIILKLIKSLQTQKLLAERLKTLLKVLEISQDSGADDKQVKKLLDEIRKLVEKIEKLARKQTKLVEKLLKKGPGNDIILKLIKSLQTQKLLAERLKTLLKVLEISQDSGADDKQVKKLLDEIRKLVEKIEKLARKQTKLVEKLLKKD

SEQ ID NO: 5 - protein, 4 aa, Artificial Sequence
  Linker
GSGN

SEQ ID NO: 6 - protein, 4 aa, Artificial Sequence
  Linker
GPGN

SEQ ID NO: 7 - protein, 152 aa, Artificial Sequence
  Synthetic polypeptide
MGSRTMYIRALEQSLREQEELAKRLKELLRELERLQREGSSDRDVKVLLWEIEALVEEIEKLARLQKELVEKLKRQGSGNMYIRALEQSLREQEELAKRLKELLRELERLQREGSSDRDVKVLLWEIEALVEEIEKLARLQKELVEKLKRQD

SEQ ID NO: 8 - protein, 151 aa, Artificial Sequence; identical to SEQ ID NO: 4
  Synthetic polypeptide

SEQ ID NO: 9 - protein, 76 aa, Artificial Sequence; identical to SEQ ID NO: 1
  Synthetic polypeptide

(19) United States
(12) Patent Application Publication
(10) Pub. No.: US 2022/0162265 A1
(43) Pub. Date: May 26, 2022

(54) SELF-ASSEMBLING 2D ARRAYS WITH DE NOVO PROTEIN BUILDING BLOCKS

(71) Applicant: UNIVERSITY OF WASHINGTON, Seattle, WA (US)

(72) Inventors: Zibo CHEN, Seattle, WA (US); David BAKER, Seattle, WA (US); Frank DIMAIO, Seattle, WA (US)

(21) Appl. No.: 17/598,641

(22) PCT Filed: Apr. 14, 2020
(86) PCT No.: PCT/US2020/028044

§ 371 Date: Sep. 27, 2021

Related U.S. Application Data
(60) Provisional application No. 62/833,902, filed on Apr. 15, 2019.

(51) Int. Cl. C07K 14/00; G16B 15/20; G16B 20/30; C12N 15/70

(52) U.S. Cl. CPC C07K 14/001; C12N 15/70; G16B 20/30; G16B 15/20

(57) ABSTRACT

Disclosed herein are polypeptides that serve as building blocks that can be used, for example, to design 2D protein arrays, methods for designing such polypeptides, and methods for their use.

### CROSS REFERENCE

This application claims priority to U.S. Provisional Application Ser. No. 62/833,902 filed Apr. 15, 2019, incorporated by reference herein in its entirety.

### FEDERAL FUNDING STATEMENT

This invention was made with government support under Grant No. GM123089, awarded by the National Institutes of Health. The government has certain rights in the invention.

### REFERENCE TO SEQUENCE LISTING

This application contains a Sequence Listing submitted as an electronic text file named “19-599-PCT_Sequence-Listing_ST25.txt”, having a size in bytes of 9 kb, and created on Apr. 6, 2020. The information contained in this electronic file is hereby incorporated by reference in its entirety pursuant to 37 CFR § 1.52(e)(5).

### BACKGROUND

Modular self-assembly of biomolecules in two dimensions (2D) is straightforward with DNA but is difficult to realize with proteins, due to the lack of modular specificity similar to Watson-Crick base pairing. The design of building blocks to enable programmable protein self-assembly is thus of importance.

### SUMMARY

In a first aspect, the disclosure provides polypeptides comprising the amino acid sequence at least 60%, 65%, 70%, 75%, 80%, 85%, 90%, 91%, 92%, 93%, 94%, 95%, 96%, 97%, 98%, 99%, or 100% identical along the full length of the amino acid sequence selected from the group consisting of:

2D-HBN_Homo
(SEQ ID NO: 1)
(GELT)DIILKLIKSLQTQKLLAERLKTLLK
VLEISQDSGADDKQVKKLLDEIRKLVEKIEK
LARKQTKLVEKLLKK(D);
and
2D-HP_Homo
(SEQ ID NO: 2)
(SRT)MYIRALEQSLREQEELAKRLKELLRE
LERLQREGSSDRDVKVLLWEIEALVEEIEKL
ARLQKELVEKLKRQ,

wherein (i) residues in parentheses are optional, and (ii) at least 1 of the highlighted residues is invariant.

In one embodiment, the polypeptide comprises two copies of SEQ ID NO:1 or SEQ ID NO:2 connected by a linking peptide. In one embodiment, the linking peptide is between 3-6 amino acids in length.

In a further embodiment, the polypeptide comprises the amino acid sequence at least 60%, 65%, 70%, 75%, 80%, 85%, 90%, 91%, 92%, 93%, 94%, 95%, 96%, 97%, 98%, 99%, or 100% identical along the full length of the amino acid sequence selected from the group consisting of:

>2D-HP
(SEQ ID NO: 3)
SRTMYIRALEQSLREQEELAKRLKELLRELE
RLQREGSSDRDVKVLLWEIEALVEEIEKLAR
LQKELVEKLKRQGSGNMYIRALEQSLREQEE
LAKRLKELLRELERLQREGSSDRDVKVLLWE
IEAIVEEIEKLARLQKELVEKLKRQD;
and
>2D-HEN
(SEQ ID NO: 4)
GELTDIILKLIKSLQTQKLLAERLKTLLKVL
EISQDSGADDKQVKKLLDEIRKLVEKIEKLA
RKQTKLVEKLLKKGPGNDII [symbol] LIKSLQTQK
LLAERLKTLLKVLEISQDSGADDKQVKKLLD
EIRKLVEKIEKLARKQTKLVEKLLKKD;

wherein at least 1 of the highlighted residues is invariant.

In one embodiment, the polypeptide comprises the amino acid sequence at least 60%, 65%, 70%, 75%, 80%, 85%, 90%, 91%, 92%, 93%, 94%, 95%, 96%, 97%, 98%, 99%, or 100% identical along the full length of SEQ ID NO:3, and wherein at least 1, 2, 3, 4, 5, 6, 7, 8, 9, 10, 11, 12, 13, 14, 15, 16, 17, 18, 19, 20, 21, 22, 23, or all 24 of the highlighted residues are invariant. In another embodiment, the polypeptide comprises the amino acid sequence at least 60%, 65%, 70%, 75%, 80%, 85%, 90%, 91%, 92%, 93%, 94%, 95%, 96%, 97%, 98%, 99%, or 100% identical along the full length of SEQ ID NO:4, and wherein at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, or all 68 of the highlighted residues are invariant.

In another embodiment, the polypeptides further comprise one or more functional domains. In a further embodiment, the disclosure provides two-dimensional assemblies, comprising a plurality of assembled polypeptides according to any embodiment or combination of embodiments of the disclosure.

In other aspects, the disclosure provides nucleic acids encoding the polypeptide of any embodiment or combination of embodiments of the disclosure, expression vectors comprising the nucleic acids operatively linked to a control sequence, and recombinant host cells comprising the nucleic acid and/or the expression vector of the disclosure.

Also disclosed are uses of the polypeptides and two-dimensional assemblies of the disclosure, and method for designing polypeptides that can form two-dimensional arrays.

### DESCRIPTION OF THE FIGURES

FIG. 1A-G. Overview of the design concept. (A) 2D self-assembly using homodimers as building blocks. Designed inter-building block binding interfaces are highlighted. In this scenario the assembly process will result in an infinite 2D lattice. (B) By using designed loops to monomerize the building block, and modularly mixing orthogonal interfaces, programmatic assembly design is enabled (in this case, a heterotetramer). (C)-(G), Overview of the design process. A de novo designed homodimer (C) is connected into a single chain (D), and docked in a C 1 2 layer group symmetry with three parameters a, b, and θ (E), resulting in a 2D lattice (F). Binding interfaces are designed with hydrogen bond networks to confer specificity (G). (H) A 1.74 Å resolution crystal structure of the design SC_2L4HC2_23 (PDB ID 6EGC) superimposed onto the design model; the design model deviates from the crystal by 1.08 Å RMSD.

FIG. 2A-C. Structural analysis of the designed 2D assembly 2D-HP. (A) Lattice design of 2D-HP, with the black box showing unit cell. (B) Designed interface of 2D-HP with exclusive hydrophobic packing across the interface. (C) Negatively stained array of 2D-HP under electron microscopy. All scale bars: black, 5 nm; white, 50 nm.

FIG. 3A-C. Structural analysis of the designed 2D assembly 2D-HBN. (A) Lattice design of 2D-HBN, and its designed interface with a hydrogen bond network (B). (C) Negatively stained array of 2D-HBN showing an extensive and flexible 2D assembly.

FIG. 4A-C. Design 2D-HP displayed two distinct morphologies under different staining solutions. (A) Thick, bundle-like structures formed in the uranyl acetate staining solution, likely due to the overall flexibility of designed 2D assemblies. (B) Individual fiber-like structures can be seen in the nanoW™ staining solution. (C) CD spectra for the thermal denaturation of 2D-HP. Wavelength scans were performed at 25° C., 75° C., 95° C., and final 25° C. Design was alpha helical and stable up to 95° C.

FIG. 5A-B. Comparison of 2D-HBN lattices using the monomer SC_2L4HC2_23 (A) or the homodimer 2L4HC2_23 (B) as building blocks. Negative stain EM shows similar patterns of 2D lattice formation in both cases. Scale bar: 50 nm.

FIG. 6A-C. 2D class average of 2D-HBN. (A) Representative image of 2D-HBN used for 2D class averaging. Inset on the right represents one of the 1,893 boxed sections picked for 2D averaging. (B) 2D class average of 2D-HBN assembly with homodimer building blocks. (C) Fourier transform of the 2D class average in (B).

FIG. 7A-D. Docking the building block into observed lattice dimensions of 68 and 22 Å results in clashes. Black box marks the unit cell, with dimensions shown outside the box. A)-D): 0°, 45°, 90°, and 135° rotation of the building block around its central axis.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise. All embodiments of any aspect of the disclosure can be used in combination, unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

In one aspect, the disclosure provides polypeptides comprising the amino acid sequence at least 60%, 65%, 70%, 75%, 80%, 85%, 90%, 91%, 92%, 93%, 94%, 95%, 96%, 97%, 98%, 99%, or 100% identical along the full length of the amino acid sequence selected from the group consisting of:

2D-HBN_Homo
(SEQ ID NO: 1)
(GELT)DIILKLIKSLQTQKLLAERLKTLLK
VLEISQDSGADDKQVKKLLDEIRKLVEKIEK
LARKQTKLVEKLLKK(D);
and
>2D-HP_Homo
(SEQ ID NO: 2)
(SRT)MYIRALEQSLREQEELAKRLKELLRE
LERLQREGSSDRDVKVLLWEIEALVEEIEKL
ARLQKELVEKLKRQ,

wherein (i) residues in parentheses are optional, and (ii) at least 1 of the highlighted residues is invariant.

As disclosed herein, the inventors have designed polypeptide building blocks that can be used, for example, to design 2D protein arrays. In this embodiment, the polypeptides are monomeric building blocks for the polypeptides that can form the 2 D arrays, and have been designed as detailed in the attached appendices.

As described in the examples, the polypeptides can tolerate significant substitutions, particularly in the non-highlighted residues. In some embodiments, a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Polypeptides comprising conservative amino acid substitutions can be tested in any one of the assays described herein to confirm that the desired activity is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In all of these embodiments, the percent identity requirement does not include any additional functional domain that may be incorporated in the polypeptide.

In one embodiment, the polypeptide comprises the amino acid sequence at least 60%, 65%, 70%, 75%, 80%, 85%, 90%, 91%, 92%, 93%, 94%, 95%, 96%, 97%, 98%, 99%, or 100% identical along the full length of SEQ ID NO:1, and wherein at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or all 34 of the highlighted residues are invariant. In another embodiment, the polypeptide comprises the amino acid sequence at least 60%, 65%, 70%, 75%, 80%, 85%, 90%, 91%, 92%, 93%, 94%, 95%, 96%, 97%, 98%, 99%, or 100% identical along the full length of SEQ ID NO:2, and wherein at least 1, 2, 3, 4, 5, 6, 7, 8, 9, 10, 11, or all 12 of the highlighted residues are invariant.

The highlighted residues include residues present at interfaces that participate in protein-protein interactions and residues designed to provide additional hydrogen bonding, as detailed in the examples that follow.

The polypeptides may comprise multiple (2, 3, 4, 5, 6, 7, 8, 9, 10, or more) copies, connected by a linking peptide. Such constructs are particularly useful, for example, in serving as scaffolds for electronic microscopy (such as cryo-EM) structure determination. In one embodiment, the polypeptide comprises two copies of SEQ ID NO:1 connected by a linking peptide. In another embodiment, the polypeptide comprises two copies of SEQ ID NO:2 connected by a linking peptide. Any suitable linking peptides may be used as deemed appropriate for given purpose. The linking peptide may be of any suitable amino acid composition and/or length. In one non-limiting embodiment, the linking peptide is between 3-6 amino acids in length. Exemplary such linking peptides include, but are not limited to, GSGN (SEQ ID NO:5) and GPGN (SEQ ID NO:6)

In another embodiment, the polypeptide comprises the amino acid sequence at least 60%, 65%, 70%, 75%, 80%, 85%, 90%, 91%, 92%, 93%, 94%, 95%, 96%, 97%, 98%, 99%, or 100% identical along the full length of the amino acid sequence selected from the group consisting of:

>2D-HP
(SEQ ID NO: 3)
SRTMYIRALEQSLREQEELAKRLKELLRELE
RLQREGSSDRDVKVLLWEIEALVEEIEKLAR
LQKELVEKLKRQGSGNMYIRALEQSLREQEE
LAKRLKELLRELERLQREGSSDRDVKVLLWE
IEAIVEEIEKLARLQKELVEKLKRQD;
and
>2D-HEN
(SEQ ID NO: 4)
GELTDIILKLIKSLQTQKLLAERLKTLLKVL
EISQDSGADDKQVKKLLDEIRKLVEKIEKLA
RKQTKLVEKLLKKGPGNDII [symbol] LIKSLQTQK
LLAERLKTLLKVLEISQDSGADDKQVKKLLD
EIRKLVEKIEKLARKQTKLVEKLLKKD;

wherein at least 1 of the highlighted residues is invariant.

In one embodiment, the polypeptide comprises the amino acid sequence at least 60%, 65%, 70%, 75%, 80%, 85%, 90%, 91%, 92%, 93%, 94%, 95%, 96%, 97%, 98%, 99%, or 100% identical along the full length of SEQ ID NO:3, and wherein at least 1, 2, 3, 4, 5, 6, 7, 8, 9, 10, 11, 12, 13, 14, 15, 16, 17, 18, 19, 20, 21, 22, 23, or all 24 of the highlighted residues are invariant.

In another embodiment, the polypeptide comprises the amino acid sequence at least 60%, 65%, 70%, 75%, 80%, 85%, 90%, 91%, 92%, 93%, 94%, 95%, 96%, 97%, 98%, 99%, or 100% identical along the full length of SEQ ID NO:4, and wherein at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, or all 68 of the highlighted residues are invariant.

As used throughout the present application, the term “polypeptide” is used in its broadest sense to refer to a sequence of subunit amino acids. The polypeptides of the invention may comprise L-amino acids, D-amino acids (which are resistant to L-amino acid-specific proteases in vivo), or a combination of D- and L-amino acids. The polypeptides described herein may be chemically synthesized or recombinantly expressed. The polypeptides may be linked to other compounds to promote an increased half-life in vivo, such as by PEGylation, HESylation, PASylation, glycosylation, or may be produced as an Fc-fusion or in deimmunized variants. Such linkage can be covalent or non-covalent as is understood by those of skill in the art.

As will be understood by those of skill in the art, the polypeptides of the invention may include additional residues at the N-terminus, C-terminus, or both that are not present in the polypeptides disclosed herein; these additional residues are not included in determining the percent identity of the polypeptides of the invention relative to the reference polypeptide.

In one embodiment, the polypeptides may further comprise one or more functional domains. As used herein, a “functional domain” is any polypeptide of interest that might be fused or covalently bound to the polypeptides of the disclosure. In non-limiting embodiments, such functional domains may comprise one or more polypeptide antigens, polypeptide therapeutics, enzymes, detectable domains, etc. The one or more functional domains may be fused at any appropriate regions within the polypeptides of the disclosure, including but not limited to at the N-terminus or at the C-terminus of the polypeptide.

As described in the examples that follow, the polypeptides of the disclosure are polypeptide building blocks that can be used, for example, to design 2D protein arrays. Thus, in another embodiment, the disclosure provides two-dimensional assemblies, comprising a plurality of assembled polypeptides according to any embodiment or combination of embodiments disclosed herein. In one embodiment, the two-dimensional assemblies may comprise a plurality of functional domains present on the assembly, via covalent or non-covalent attachment. Non-limiting and exemplary such functional domains are described above.

The polypeptides and two-dimensional assemblies can be used for any suitable purpose. In various embodiments, they may be used as scaffolds on which to fuse antigens to increase immune response due to the increase in avidity; as scaffolds for structure determination by cryo EM or X-ray crystallography when fused to proteins of interest; as a platform for the construction of molecular robots due to the regular spacing of the assemblies; as a surface for construction of regularly spaced enzyme assembly lines; or as protein materials for coating purposes.

In another aspect, the disclosure provides nucleic acids encoding the polypeptide of any embodiment or combination of embodiments of the disclosure. The nucleic acid may comprise single stranded or double stranded RNA or DNA, or DNA-RNA hybrids, each of which may include chemically or biochemically modified, non-natural, or derivatized nucleotide bases. Such nucleic acids may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In another aspect, the disclosure provides expression vectors comprising the nucleic acids of the disclosure operatively linked to a control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the nucleic acids and/or or expression vectors (i.e.: episomal or chromosomally integrated) disclosed herein, wherein the host cells can be either prokaryotic or eukaryotic. The cells can be transiently or stably engineered to incorporate the expression vector of the disclosure, using techniques including but not limited to bacterial transformations, calcium phosphate co-precipitation, electroporation, or liposome mediated-, DEAE dextran mediated-, polycationic mediated-, or viral mediated transfection.

In a further aspect, the disclosure provides methods for designing polypeptides that can form two-dimensional arrays, comprising any method as described in the attached examples. In one embodiment, the method comprises

(a) modifying a polypeptide that forms a homodimer by adding a loop sequence to link two copies of the monomeric polypeptide to form a building block;

(b) docking the building block into pseudo-C 1 2 layer group and systematically sampling three parameters that control lattice geometry: two parameters describing the lattice dimensions, and one parameter controlling rotation of the building block around its central axis; and

(c) computationally modifying interface residues and enhancing binding specificity between monomers by designing buried hydrogen bond networks at the interface between subunits, selecting for networks that involve at least 3 side chain residues with all heavy-atom donors and acceptors participating in hydrogen bonds.

The design methods are described in detail in the examples, and all embodiments or combinations of embodiments disclosed therein may be used in the design methods of the disclosure.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

### Examples

Modular self-assembly of biomolecules in two dimensions (2D) is straightforward with DNA but has been difficult to realize with proteins, due to the lack of modular specificity similar to Watson-Crick base pairing. Here we describe a general approach to design 2D arrays using de novo designed pseudosymmetric protein building blocks. A homodimeric helical bundle was reconnected into a monomeric building block, and the surface was redesigned in Rosetta™ to enable self-assembly into a 2D array in the C 1 2 layer symmetry group. The designed arrays assembled to sub-μm scale under both negative stain electron microscopy and atomic force microscopy, and displayed the designed lattice geometry. The design of 2D arrays with pseudosymmetric building blocks is an important step toward the design of programmable protein self-assembly via pseudosymmetric patterning of orthogonal binding interfaces.

### INTRODUCTION

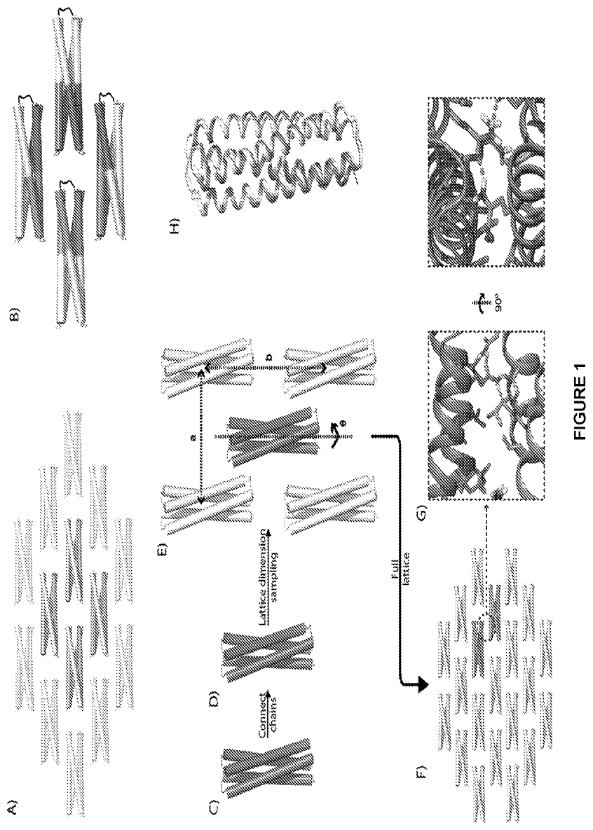

Here we describe a general approach for generating pseudosymmetric 2D assemblies based on a C 1 2 symmetric layer group. Starting from a de novo designed homodimer, we first design a new loop to monomerize the backbone of our building block, then identify configurations of this backbone capable of forming 2D arrays with pseudo-C 1 2 symmetry (The resulting layer group symmetry is pseudo-C 1 2 because each building block has pseudo-C2 symmetry due to the presence of an additional loop), and finally redesign the interface so that the building block will be programmatically assembled into 2D arrays with the prescribed unit cell dimensions and subunit configuration. This monomerization of the multimeric protein building block allows unique sequences to be designed on each of the 4 binding interfaces, ultimately enabling the modular assembly of higher order interactions through the design of mutually orthogonal interfaces with the same subunit placement and unit cell dimensions (FIG. 1 A, B). This study helps enable various applications including patterned enzymatic reactions.

### Results

We developed a general strategy for the design of pseudosymmetric 2D protein assemblies using de novo designed proteins as building blocks, fully described in Methods. FIG. 1 presents a high-level overview of the approach. Briefly, a previously designed helical bundle homodimer 2L4HC2_23 (PDB ID 5J0K (12), FIG. 1C) was connected into a single chain monomer via a designed loop, resulting in a pseudo-C2 symmetric building block (SC_2L4HC2_23, FIG. 1D). We solved the X-ray crystal structure of the building block, revealing a backbone nearly identical to the design model and the original 2L4HC2_23 homodimer structure, with a Ca root mean square deviation (RMSD) of 1.08 Å between the design and crystal structure (FIG. 1H).

Using this monomerized building block as a starting point for pseudosymmetric assembly, we subsequently enumerated all possible pseudo-C 1 2 symmetric layer assemblies compatible with this design, exhaustively sampling three degrees of freedom: two parameters describing the lattice dimensions, and one parameter controlling rotation of the building block around its central axis (FIG. 1E). We sampled 576,000 settings of these three parameters, and removed those which were not capable of forming a connected, non-clashing 2D assembly (FIG. 1F). The remaining ˜1,000 designs had their surfaces redesigned to self-assemble into the corresponding lattice arrangement using standard Rosetta™ fixed backbone design (15). Using computationally predicted interface energies as well as visual inspection, seven designs were selected for experimental characterization.

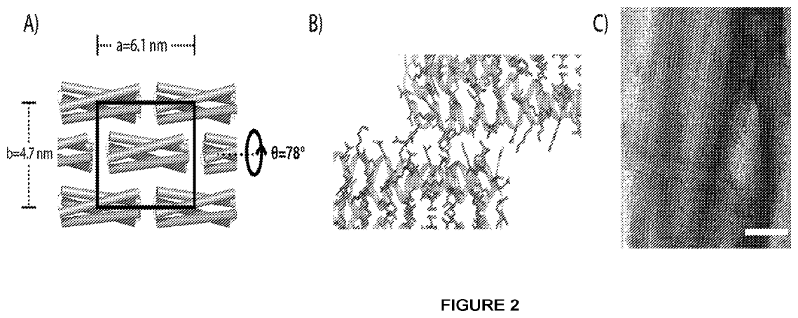

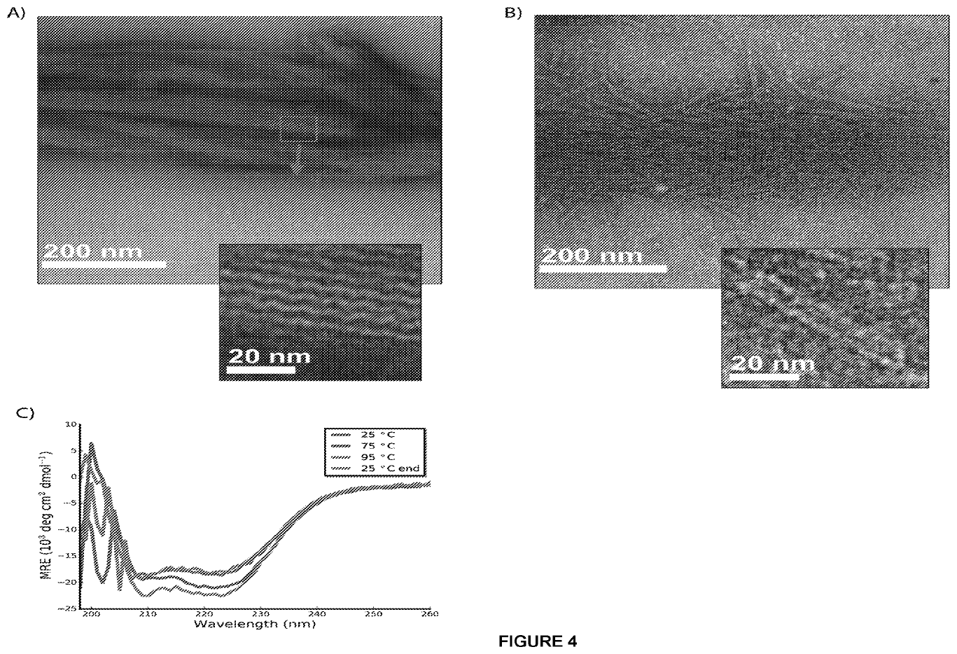

Examination by negative-stain electron microscopy (EM) and atomic force microscopy (AFM) revealed regular arrays on the sub-μm scale for one of the designs with exclusively hydrophobic residues at the binding interfaces (2D-HP, FIG. 2 A, B). The design showed α-helical characteristics and was stable up to 950 (FIG. 4 C) as measured by circular dichroism (CD). Negative-stain EM revealed the clustering of 2D arrays into bundle-like structures that are sensitive to different staining molecules (FIG. 2C, FIG. 4 A-B).

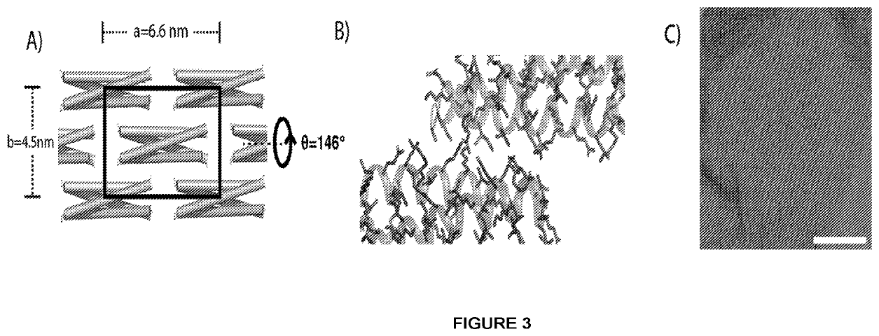

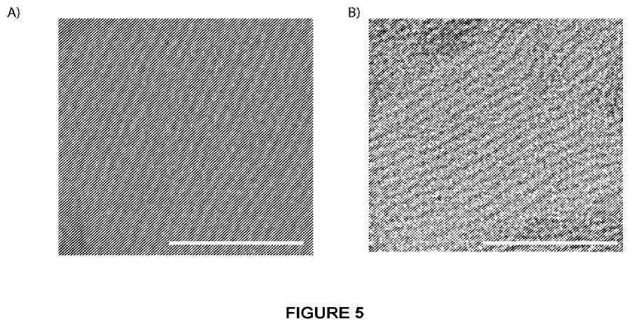

Given the non-specific clustering of 2D-HP assemblies under EM, we sought to further improve the binding specificity among building blocks by designing buried hydrogen bonds at the interface (FIG. 1G). A systematic search of interfacial hydrogen bond networks on 576,000 lattice dimensions resulted in 24 designs with no buried unsatisfied polar heavy atoms and good interfacial binding energy. After a round of in silico selection, three such designs were ordered, with one of the designs (2D-HBN, FIG. 3 A, B) forming more extended and regular assemblies compared to that of 2D-HP (FIG. 3C), likely due to better binding specificity conferred by hydrogen bond networks. To rule out the possibility of domain swapping from the single chain building block contributing to the final assembly, we additionally expressed the building block protein of 2D-HBN as individual homodimers of helix hairpins, which similarly assembled into 2D arrays of the same morphology under the same condition (FIG. 5).

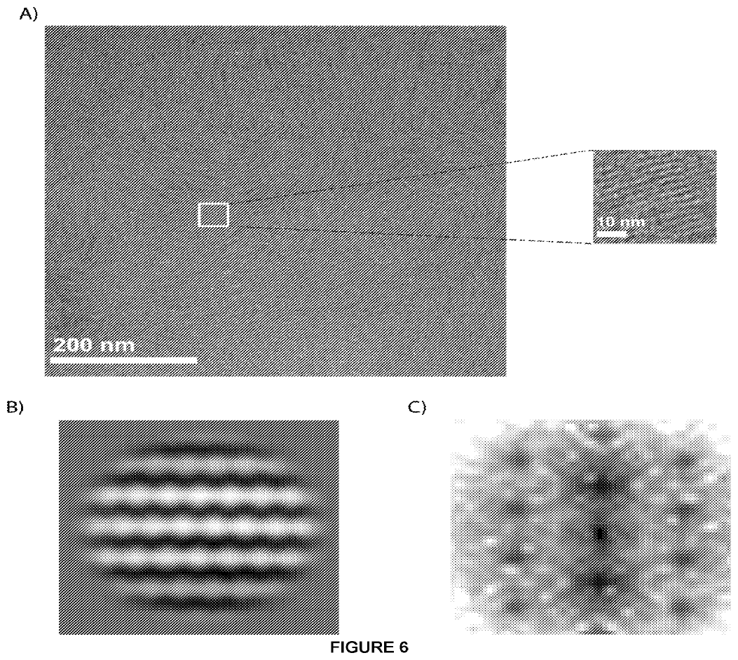

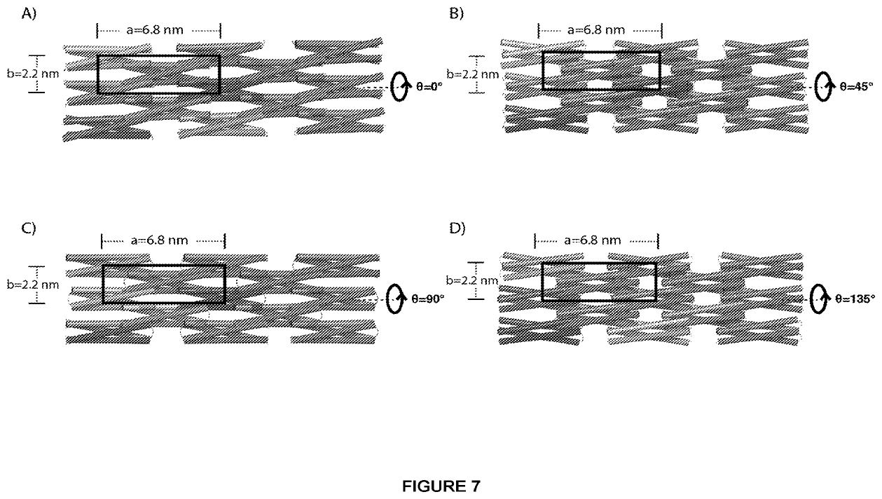

To verify that the array was forming a regular 2D grid, we collected a larger negative stain (NanoW™) dataset of the best-behaved arrays (FIG. 6 A). Subsequent 2D classification and averaging of 1,893 boxed ˜20 nm regions yielded an image showing an ordered two-dimensional assembly with a power spectrum indicating first-order spots (FIG. 6 B-C). While the resulting images were consistent with a C 1 2-symmetric complex, the unit-cell dimensions were somewhat different than designed: while the design had a 6.6 by 4.5 nm unit cell, the experimental images indicated approximately a 6.8 by 2.2 nm unit cell. Given the inability to pack the designed model into this observed space group (FIG. 7).

### DISCUSSION

We showed that by systematically sampling lattice dimensions followed by computational interface design, the same de novo designed helical bundle building block can be modularly self-assembled into two arrays with unique cell dimensions. As more de novo building blocks are designed, particularly with higher-order symmetry, a variety of 2D assemblies with unique layer group symmetries are achievable with the same design protocol, including those using larger de novo building blocks, and designing in non-polar layer groups, which have a rotation about the layer plane (e.g., P 3 2 1 and P 4 2₁ 2), effectively canceling out any “curvature” errors in binding along the z axis, further flattening out the 2D assembly.

The monomerization of the homodimer building block coupled with designed hydrogen bond networks allows orthogonal interfaces to be designed at each intermolecular binding site, paving the way for the programmatic self-assembly of proteins into finite shapes (FIG. 1 B), which requires the design of multiple such interfaces on a single configuration of a building block. Such interfaces can be applied modularly, by plugging designed sequence on to the corresponding helical bundle. Our work represents a key step toward this goal and shows that de novo designed proteins can serve as building blocks for 2D assemblies.

### REFERENCES

1. Takenoya M, Nikolakakis K, Sagermann M (2010) Crystallographic insights into the pore structures and mechanisms of the EutL and EutM shell proteins of the ethanolamine-utilizing microcompartment of Escherichia coli. J Bacteriol 192(22):6056-6063.
2. Guo F, et al. (2014) Capsid expansion mechanism of bacteriophage T7 revealed by multistate atomic models derived from cryo-EM reconstructions. Proc Natl Acad Sci USA 111(43):E4606-14.
3. Klug A (1999) The tobacco mosaic virus particle: structure and assembly. Philos Trans R Soc Lond B Biol Sci 354(1383):531-535.
4. Rothemund P W K (2006) Folding DNA to create nanoscale shapes and patterns. Nature 440(7082):297-302.
5. Wei B, Dai M, Yin P (2012) Complex shapes self-assembled from single-stranded DNA tiles. Nature 485(7400):623-626.
6. Tikhomirov G, Petersen P, Qian L (2017) Programmable disorder in random DNA tilings. Nat Nanotechnol 12(3):251-259.
7. Gonen S, DiMaio F, Gonen T, Baker D (2015) Design of ordered two-dimensional arrays mediated by noncovalent protein-protein interfaces. Science 348(6241):1365-1368.
8. Suzuki Y, et al. (2016) Self-assembly of coherently dynamic, auxetic, two-dimensional protein crystals. Nature 533(7603):369-373.
9. Liu Y, Gonen S, Gonen T, Yeates T O (2018) Near-atomic cryo-EM imaging of a small protein displayed on a designed scaffolding system. Proc Natl Acad Sci USA. doi:10.1073/pnas.1718825115.
10. Jiang T, Xu C, Zuo X, Conticello V P (2014) Structurally homogeneous nanosheets from self-assembly of a collagen-mimetic peptide. Angew Chem Int Ed Engl 53(32):8367-8371.
11. Fletcher J M, et al. (2012) A basis set of de novo coiled-coil peptide oligomers for rational protein design and synthetic biology. ACS Synth Biol 1(6):240-250.
12. Boyken S E, et al. (2016) De novo design of protein homo-oligomers with modular hydrogen-bond network-mediated specificity. Science 352(6286):680-687.
13. Fallas J A, et al. (2017) Computational design of self-assembling cyclic protein homo-oligomers. Nat Chem 9(4):353-360.
14. Huang P-S, et al. (2014) High thermodynamic stability of parametrically designed helical bundles. Science 346(6208):481-485.
15. Leaver-Fay A, et al. (2011) ROSETTA3: an object-oriented software suite for the simulation and design of macromolecules. Methods Enzymol 487:545-574.
16. Gray J J, et al. (2003) Protein-protein docking with simultaneous optimization of rigid-body displacement and side-chain conformations. J Mol Biol 331(1):281-299.

### Supplemental Materials

### Program Code

Step 1. Rapid Generation of Connecting and Non-Clashing 2D Lattices from Protein Building Blocks
˜/Rosetta/main/source/bin/flatland.static.linuxgccrelease
-in:file:s [input pdb model]
-database [path to Rosetta database]
-ignore_unrecognized_res
-mh:path:scores_BB_BB
/gscratch/baker/zibochen/utilities/aa_count_ACDEFHIKLMNQRSTVWY_resl1_ang15_msc 0.2_smooth1.3_ROSETTA/aa_count_ACDEFHIKLMNQRSTVWY_resl1_ang15_msc0.2_smooth1.3_ROSETTA-mh:score:use_ss1 true-mh:score:use_ss2 true-mh:score:use_aa1 false
-mh:score:use_aa2 false #motif score specific options
-symmetry_definition dummy
-output_virtual
-tag [user defined name tag for the job]
-rot_step [search step size for the self-rotation of the building block, takes a real number]
-Cn [internal cyclic symmetry of the building block, 2]
-wallpaper [layer symmetry of the final 2D lattice, C211]
-dump_silent [dump a silent file containing all the lattices, boolean]
-C21_B [lattice parameter B for the C 1 2 layer group, takes a real number]
-cell_upper [upper limit for the cell dimensions, takes a real number]
-single_chain_version [if the input model is monomerized, the code accommodates for this psudeo-symmetry. Boolean]
-cell_step [search step size for the lattice cell dimensions, takes a real number]

### Step 2. HBNet Search at the Interfaces of Extracted Adjacent Building Blocks

˜/Rosetta/main/source/bin/rosetta_scripts.static.linuxgccrelease
-in:file:s [input pdb model]
-out::file::pdb_comments
-run:preserve_header
-use_input_sc
-out:prefix HBNet_
-beta
-missing_density_to_jump true
-parser:protocol 2D_HBNet.xml
-database [path to Rosetta database]
-chemical:exclude_patches LowerDNA UpperDNA Cterm_amidation VirtualBB ShoveBB VirtualDNAPhosphate VirtualNTerm CTermConnect sc_orbitals pro_hydroxylated_case1 pro_hydroxylated_case2 ser_phosphorylated thr_phosphorylated tyr_phos phorylated tyr_sulfated lys_dimethylated lys_monomethylated lys_trimethylated lys_acetylated glu_carboxylated cys_acetylated tyr_diiodinated N_acetylated C_methylamidated MethylatedProteinCterm
-in:file:fullatom
-multi_cool_annealer 10
-no_optH false
-optH_MCA true
-flip_HNQ

### Step 3. Regenerate the Complete 2D Lattice and Map Newly Designed Interfaces to all Symmetric Copies

˜/Rosetta/main/source/bin/symm_seq_gen_2D.default.linuxgccrelease
-database [path to Rosetta database]
-s [input pdb model]
-cn [symmetry of the building block, 2]

### Step 4. Symmetric Design of the 2D Lattice in the Context of its Symmetry

˜/Rosetta/main/source/bin/symm_seq_gen_2D.default.linuxgccrelease
-database [path to Rosetta database]\-in:file:silent [input Rosetta silent file containing the 2D lattice]
-parser:script_vars resfile=[input resfile to enfore newly designed interfaces stay intact]
-out::file::pdb_comments
-run:preserve_header
-multi_cool_annealer 10
-use_input_sc
-symmetry_definition dummy
-out:prefix packed_
-beta-missing_density_to_jump true
-symmetry:detect_bonds false
-parser:protocol 2D_final_design.xml

### Supplemental Materials

### Computational Design Methods

1. Connecting the Homodimer into Monomer

The two monomers from the homodimer 2L4HC2_23 are connected into a single chain monomer with a 5-residue loop. Briefly, a database of backbone samples composed of fragments spanning two helical regions via a loop of five or less residues was generated from high resolution crystallographic structures. Loops in this database were then structurally aligned to terminal residues of the design backbone, and those that aligned within 0.35 Å RMSD were carried forward with full Rosetta design restricted to the loop and its neighborhood residues within 6 Å. The lowest-scoring candidate selected as the final loop design.

### 2. Systematic Sampling of Lattice Parameters

A custom Rosetta protocol was developed to dock the building block into pseudo-C 1 2 layer group and systematically sample the three parameters that control lattice geometry: two parameters describing the lattice dimensions, and one parameter controlling rotation of the building block around its central axis (FIG. 1E). Taking into account the dimension of the building block, lattice parameter “a” was sampled from 60 Å to 100 Å, with a step size of 0.5 Å; lattice parameter “b” was sampled from 30 Å to 50 Å, with a step size of 0.5 Å; rotation of the building block around its central axis, θ, was sampled from 0° to 180° with a step size of 1°, resulting in 576,000 possible docked conformations. A rapid evaluation protocol in Rosetta™ was applied to remove lattices that have either clashes of building blocks or inter building block distance greater than 10 Å, resulting in 4,139 candidate lattices for further design. Two adjacent building blocks were extracted from the lattice for interface design calculations.

### 3. Design Calculations

RosettaDesign™ calculations were carried out on the interfaces between adjacent building blocks, while keeping the rest of the sequences fixed. To enhance the binding specificity among subunits, we optionally used the Rosetta™ HBNet™ algorithm to design buried hydrogen bond networks at the interface between subunits, selecting for networks that involve at least 3 side chain residues with all heavy-atom donors and acceptors participating in hydrogen bonds. Low energy sequences were identified using RosettaDesign™ calculations in which the hydrogen bond networks were held fixed. A final step of minimization and side chain repacking without atom pair constraints was applied to identify the movement of HBNets™, filtering out designs with significantly moved HBNets™. The complete 2D lattice was then regenerated using the adjacent building blocks (now with designed interfaces), with the newly designed sequences applied to all building blocks. A final round of Rosetta™ design was carried out in the context of the C 1 2 layer group symmetry with the newly designed sequences fixed, to resolve potential side chain clashes in the final lattice.

### 4. Selection Criteria and Metrics Used to Evaluate Designs

Fully designed models were selected based on the shape-complementarity of the designed interface (SC>0.6), size of the designed interfaces (dSASA>500 Å), average binding energy (ddG/dSASA<−0.02 Rosetta™ Energy Unit/Å²) and no buried unsatisfied hydrogen bonds introduced at the new interfaces. Selected designs were then visually inspected for good packing of hydrophobic side chains at the interfaces.

### Visualization and Figures

All structural images for figures were generated using PyMOL (3).

### Experimental Methods

### Buffer and Media Recipe

### TBM-5052

1.2% [wt/vol] tryptone, 2.4% [wt/vol] yeast extract, 0.5% [wt/vol] glycerol, 0.05% [wt/vol]D-glucose, 0.2% [wt/vol] D-lactose, 25 mM Na2HPO4, 25 mM KH2PO4, 50 mM NH4Cl, 5 mM Na2SO4, 2 mM MgSO4, 10 μM FeCl3, 4 μM CaCl₂), 2 μM MnCl2, 2 μM ZnSO4, 400 nM CoCl2, 400 nM NiCl2, 400 nM CuCl2, 400 nM Na2MoO4, 400 nM Na2SeO3, 400 nM H3BO3

### TBS Buffer

20 mM Tris pH 8.0, 100 mM NaCl

### Construction of Synthetic Genes

Synthetic genes were ordered from Genscript Inc. (Piscataway, N.J., USA) and delivered in pET28b(+) E. coli expression vector, inserted between the NdeI and XhoI sites.

### Protein Expression

Plasmids were transformed into chemically competent E. coli expression strains BL21(DE3)Star (Invitrogen) for protein expression. Single colonies were picked from agar plates following transformation and growth overnight, and 5 ml starter cultures were grown at 37° C. in Luria-Bertani (LB) medium containing 100 μg/mL kanamycin with shaking at 225 rpm for 18 hours at 37° C. Starter cultures were diluted into 500 ml TBM-5052 containing 100 μg/mL kanamycin, and incubated with shaking at 225 rpm for 24 hours at 37° C.

### Protein Purification

Cells were harvested by centrifugation for 15 minutes at 5000 rcf at 4° C. and resuspended in 20 ml lysis buffer. Lysozyme, DNAse, and EDTA-free cocktail protease inhibitor (Roche) were added to the resuspended cell pellet before sonication at 70% power for 5 minutes. All 10 designs expressed and precipitated into cell pellet after clearing the cell lysate at 12,000 g for 1 hour. Pellets were twice resuspended in 10 ml TBS followed by centrifugation at 12,000 g for 20 min. The resulting pellet was resuspended in 1 M GdmHCl followed by centrifugation at 12,000 g for 20 min. The supernatant was dialyzed overnight into TBS buffer.

### Circular Dichroism (CD) Measurements

CD wavelength scans (260 to 195 nm) and temperature melts (25 to 95° C.) were performed using an AVIV model 420 CD spectrometer. Temperature melts were carried out at a heating rate of 4° C./min and monitored by the change in ellipticity at 222 nm; protein samples were diluted to 0.25 mg/mL in PBS pH 7.4 in a 0.1 cm cuvette.

### X-Ray Crystallography and Structure Determination

Crystals of SC_2L4HC2_23 were grown by mixing 0.1 ul of protein at 20 mg/ml plus 0.1 ul of crystallization condition Morpheus H9 (Molecular Dimensions, 0.1M Amino acids, 0.1M Buffer System 3 pH 8.5, 50% (v/v) Precipitant Mix 1). As this solution is already a suitable cryoprotectant, crystals were flash-frozen directly in liquid nitrogen prior to data collection. Diffraction data was collected at the Advanced Light Source, Lawrence Berkeley National Laboratory, beamline 8.2.1. Diffraction data was indexed and scaled using HKL2000 (4). Initial models were generated by the molecular-replacement method using the program PHASER™ (5) within the Phenix™ software suite (6), with the computational design serving as the search model. Efforts were made to reduce model bias by using simulated annealing and prime-and-switch phasing within Phenix.autobuild (7). Iterative rounds of manual building in COOT™ (8) and refinement in Phenix were used to produce the final model. Due to the high degree of self-similarity inherit in coiled-coil-like proteins, datasets for the reported structures suffered from a high degree of pseudo translational non-crystallographic symmetry, as report by Phenix.Xtriage™, which complicated structure refinement and may explain the higher than expected R values reported. RMSDs of bond lengths, angles and dihedrals from ideal geometries were calculated with Phenix™ (6). The overall quality of all final models was assessed using the program MOLPROBITY™ (9). Summaries of diffraction data and refinement statistics are provided in Supplementary Table 2.

### Negative Stain FM

Samples were applied to glow-discharged EM grids and stained with either uranyl acetate (UA), uranyl formate (UF) or NanoW™ (Nanoprobes, Inc, Yaphank, N.Y., USA) for screening or analysis. Data was collected using a Tecnai T12 equipped with a Gatan Orius CCD. CTF estimation was performed using GCTF (10), and all other image processing steps were completed via Relion™ 2.1 (11). For the analysis in FIG. 2G, 421 2D array segments were picked manually from 42 micrographs, and the resulting 2D class average used as a template for Relion™ autopicking, which yielded 5,823 2D array segments. After subsequent 2D classification and alignment, the dominant 2D class consisted of 1,893 array segments (each approximately 20 nm diameter).

>2D-HP
MGSRTMYIRALEQSLREQEELAKRLKELLRELER
LQREGSSDRDVKVLLWEIEALVEEIEKLARLQKE
LVEKLKRQGSGNMYIRALEQSLREQEELAKRLKE
LLRELERLQREGSSDRDVKVLLWEIEALVEEIEK
LARLQKELVEKLKRQD
(SEQ ID NO: 7): Multimer of SEQ ID NO: 2;
related to SEQ ID NO: 3
>2D-HBN
GELTDIILKLIKSLQTQKLLAERLKILLKVLEIS
QDSGADDKQVKKLLDEIRKLVEKIEKLARKQTKL
VEKLLKKGPGNDIILKLIKSLQTQKLLAERLKIL
LKVLEISQDSGADDKQVKKLLDEIRKLVEKIEKL
ARKQTKLVEKLLKKD
(SEQ ID NO: 8): Multimer of SEQ ID NO: 1;
related to SEQ ID NO: 4
>2D-HBN Homo (using the homodimer
as building block)
GELTDIILKLIKSLQTQKLLAERLKILLKVLEISQ
DSGADDKQVKKLLDEIRKLVEKIEKLARKQTKLVE
KLLKKD
(SEQ ID NO: 9): Related to SEQ ID NO: 1

TABLE 2
Data collection and refinement statistics

| | SC_2L4HC2_23 | |
|---|---|---|
| Wavelength | 0.9998 | |
| Resolution range | 21-1.74 | (1.802-1.74) |
| Space group | P 1 21 1 | |
| Unit cell | 41.253 49.36 41.239 90 | |
| | 104.303 90 | |
| Total reflections | 59303 | (5381) |
| Unique reflections | 16336 | (1241) |
| Multiplicity | 3.6 | (3.4) |
| Completeness (%) | 92.01 | (76.04) |
| Mean I/sigma(I) | 9.11 | (1.26) |
| Wilson B-factor | 36.92 | |
| R-merge | 0.05331 | (0.8799) |
| R-meas | 0.06269 | (1.034) |
| R-pim | 0.03271 | (0.5389) |
| CC1/2 | 0.998 | (0.669) |
| CC* | 1 | (0.895) |
| Reflections used in refinement | 15268 | (1241) |
| Reflections used for R-free | 1461 | (121) |
| R-work | 0.2266 | (0.3887) |
| R-free | 0.2657 | (0.4216) |
| CC(work) | 0.939 | (0.792) |
| CC(free) | 0.913 | (0.658) |

| | |
|---|---|
| Number of non-hydrogen atoms | 1185 |
| macromolecules | 1134 |
| solvent | 51 |
| Protein residues | 147 |
| RMS(bonds) | 0.019 |
| RMS(angles) | 1.45 |
| Ramachandran favored (%) | 98.6 |
| Ramachandran allowed (%) | 1.4 |
| Ramachandran outliers (%) | 0 |
| Rotamer outliers (%) | 0 |
| Clashscore | 3.62 |
| Average B-factor | 54.64 |
| macromolecules | 54.26 |
| solvent | 63.1 |
| Number of TLS groups | 4 |

### REFERENCES

1. Boyken S E, et al. (2016) De novo design of protein homo-oligomers with modular hydrogen-bond network-mediated specificity. Science 352(6286):680-687.
2. Leaver-Fay A, et al. (2011) ROSETTA3: an object-oriented software suite for the simulation and design of macromolecules. Methods Enzymol 487:545-574.
3. Schrödinger, LLC (2015) The PyMOL Molecular Graphics System, Version 1.8.
4. Otwinowski Z, Minor W (1997) Processing of X-ray diffraction data collected in oscillation mode. Methods Enymol 276:307-326.
5. McCoy A J, et al. (2007) Phaser crystallographic software. J Appl Crystallogr 40(Pt 4):658-674.
6. Adams P D, et al. (2010) PHENIX: a comprehensive Python-based system for macromolecular structure solution. Acta Crystallogr D Biol Crystallogr 66(Pt 2):213-221.
7. Terwilliger T C, et al. (2008) Iterative model building, structure refinement and density modification with the \it PHENIX AutoBuild wizard. Acta Crystallogr D Biol Crystallogr 64(1):61-69.
8. Emsley P, Cowtan K (2004) Coot: model-building tools for molecular graphics. Acta Crystallogr D Biol Crystallogr 60(Pt 12 Pt 1):2126-2132.
9. Davis I W, et al. (2007) MolProbity: all-atom contacts and structure validation for proteins and nucleic acids. Nucleic Acids Res 35(Web Server issue):W375-83.
10. Zhang K (2016) Gctf: Real-time CTF determination and correction. J Struct Biol 193(1):1-12.
11. Scheres S H W (2012) RELION: implementation of a Bayesian approach to cryo-EM structure determination. J Struct Biol 180(3):519-530.

1. A polypeptide comprising the amino acid sequence at least 60%, 65%, 70%, 75%, 80%, 85%, 90%, 91%, 92%, 93%, 94%, 95%, 96%, 97%, 98%, 99%, or 100% identical along the full length of the amino acid sequence selected from the group consisting of:
>2D-HBN_Homo
(SEQ ID NO: 1)
(GELT)DIILKLIKSLQTQKLLAERLKTLLK
VLEISQDSGADDKQVKKLLDEIRKLVEKIEK
LARKQTKLVEKLLKK(D);
and
>2D-HP_Homo
(SEQ ID NO: 2)
(SRT)MYIRALEQSLREQEELAKRLKELLRE
LERLQREGSSDRDVKVLLWEIEALVEEIEKL
ARLQKELVEKLKRQ,
wherein (i) residues in parentheses are optional, and (ii) at least 1 of the highlighted residues is invariant.

2. The polypeptide of claim 1, wherein the polypeptide comprises the amino acid sequence at least 60%, 65%, 70%, 75%, 80%, 85%, 90%, 91%, 92%, 93%, 94%, 95%, 96%, 97%, 98%, 99%, or 100% identical along the full length of SEQ ID NO:1, and wherein at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or all 34 of the highlighted residues are invariant.

3. The polypeptide of claim 1, wherein the polypeptide comprises the amino acid sequence at least 60%, 65%, 70%, 75%, 80%, 85%, 90%, 91%, 92%, 93%, 94%, 95%, 96%, 97%, 98%, 99%, or 100% identical along the full length of SEQ ID NO:2, and wherein at least 1, 2, 3, 4, 5, 6, 7, 8, 9, 10, 11, or all 12 of the highlighted residues are invariant.

4. The polypeptide of claim 1, wherein the polypeptide comprises two copies of SEQ ID NO:1 connected by a linking peptide.

5. The polypeptide of claim 1, wherein the polypeptide comprises two copies of SEQ ID NO:2 connected by a linking peptide.

6. The polypeptide of claim 4, wherein the linking peptide is between 3-6 amino acids in length.

7. The polypeptide of claim 4, wherein the polypeptide comprises the amino acid sequence at least 60%, 65%, 70%, 75%, 80%, 85%, 90%, 91%, 92%, 93%, 94%, 95%, 96%, 97%, 98%, 99%, or 100% identical along the full length of the amino acid sequence selected from the group consisting of:
>2D-HP
(SEQ ID NO: 3)
SRTMYIRALEQSLREQEELAKRLKELLRELE
RLQREGSSDRDVKVLLWEIEALVEEIEKLAR
LQKELVEKLKRQGSGNMYIRALEQSLREQEE
LAKRLKELLRELERLQREGSSDRDVKVLLWE
IEAIVEEIEKLARLQKELVEKLKRQD;
and
>2D-HEN
(SEQ ID NO: 4)
GELTDIILKLIKSLQTQKLLAERLKTLLKVL
EISQDSGADDKQVKKLLDEIRKLVEKIEKLA
RKQTKLVEKLLKKGPGNDII [symbol] LIKSLQTQK
LLAERLKTLLKVLEISQDSGADDKQVKKLLD
EIRKLVEKIEKLARKQTKLVEKLLKKD;
wherein at least 1 of the highlighted residues is invariant.

8. The polypeptide of claim 7, wherein the polypeptide comprises the amino acid sequence at least 60%, 65%, 70%, 75%, 80%, 85%, 90%, 91%, 92%, 93%, 94%, 95%, 96%, 97%, 98%, 99%, or 100% identical along the full length of SEQ ID NO:3, and wherein at least 1, 2, 3, 4, 5, 6, 7, 8, 9, 10, 11, 12, 13, 14, 15, 16, 17, 18, 19, 20, 21, 22, 23, or all 24 of the highlighted residues are invariant.

9. The polypeptide of claim 7, wherein the polypeptide comprises the amino acid sequence at least 60%, 65%, 70%, 75%, 80%, 85%, 90%, 91%, 92%, 93%, 94%, 95%, 96%, 97%, 98%, 99%, or 100% identical along the full length of SEQ ID NO:4, and wherein at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, or all 68 of the highlighted residues are invariant.

10. The polypeptide of claim 1, further comprising one or more functional domains.

11. A two-dimensional assembly, comprising a plurality of assembled polypeptides according to claim 1.

12. The two-dimensional assembly of claim 11, further comprising a plurality of functional domains present on the assembly.

13. Use of the polypeptides of claim 1 for any suitable purpose, including but not limited to, as scaffolds on which to fuse antigens to increase immune response due to the increase in avidity; as scaffolds for structure determination by cryo EM or X-ray crystallography when fused to proteins of interest; as a platform for the construction of molecular robots due to the regular spacing of the assemblies; as a surface for construction of regularly spaced enzyme assembly lines; or as protein materials for coating purposes.

14. A nucleic acid encoding the polypeptide of claim 1.

15. An expression vector comprising the nucleic acid of claim 14 operatively linked to a control sequence.

16. A recombinant host cell comprising the nucleic acid of claim 14.

17. A method for designing polypeptides that can form two-dimensional arrays, comprising any method as described herein.

18. The method of claim 17, comprising:
(a) modifying a polypeptide that forms a homodimer by adding a loop sequence to link two copies of the monomeric polypeptide to form a building block;
(b) docking the building block into pseudo-C 1 2 layer group and systematically sampling three parameters that control lattice geometry: two parameters describing the lattice dimensions, and one parameter controlling rotation of the building block around its central axis; and
(c) computationally modifying interface residues and enhancing binding specificity between monomers by designing buried hydrogen bond networks at the interface between subunits, selecting for networks that involve at least 3 side chain residues with all heavy-atom donors and acceptors participating in hydrogen bonds.
